(19) United States
(12) Patent Application Publication
(10) Pub. No.: US 2011/0184395 A1
(43) Pub. Date: Jul. 28, 2011

(54) METHOD FOR LASER CAPSULOTOMY AND LENS CONDITIONING

(75) Inventors: Georg Schuele, Menlo Park, CA (US); Neil Friedman, Menlo Park, CA (US); William Culbertson, Miami, FL (US); Barry Siebel, Pacific Palisades, CA (US); Dan Andersen, Menlo Park, CA (US)

(73) Assignee: OPTIMEDICA CORPORATION, Santa Clara, CA (US)

(21) Appl. No.: 12/978,367

(22) Filed: Dec. 23, 2010

Related U.S. Application Data
(60) Provisional application No. 61/289,837, filed on Dec. 23, 2009.

(51) Int. Cl. A61B 18/20

(52) U.S. Cl. 606/5

(57) ABSTRACT

A method of creating a capsulotomy and conditioning the crystalline lens is disclosed, wherein a laser is employed that provides improved performance by treating the capsule predominantly prior to treating the lens.

### RELATED APPLICATION DATA

The present application claims the benefit under 35 U.S.C. §119 to U.S. provisional patent application Ser. No. 61/289,837, filed Dec. 23, 2009. The foregoing application is hereby incorporated by reference into the present application in its entirety.

### FIELD OF THE INVENTION

The present invention relates to laser photomedical ophthalmic procedures and systems.

### BACKGROUND OF THE INVENTION

Cataract extraction is one of the most commonly performed surgical procedures in the world with estimates of 3.5 million cases being performed annually in the United States and 15 million cases worldwide. Modern cataract surgery is typically performed by first creating an opening in the cornea and then another in the anterior lens capsule, which is termed an anterior capsulotomy or capsulorhexis. The patient's natural crystalline lens is then typically removed by ultrasonic phacoemulsification and irrigation/aspiration methods and a synthetic foldable intraocular lens (IOL) ultimately inserted into the now empty capsular bag.

The concept of the capsulotomy is to provide a smooth continuous circular opening through which not only the phacoemulsification of the nucleus can be performed safely and easily, but also for easy insertion of the intraocular lens. It provides both clear central access for insertion, a permanent aperture for transmission of the image to the retina by the patient, and also a support of the IOL inside the remaining capsule. The capsulotomy is the most technically demanding surgical step in the cataract removal procedure. The removal of the crystalline lens is the longest and most involved surgical step in the cataract removal procedure. It typically requires the use of appreciable amounts of ultrasonic energy to fragment the lens into pieces small enough to be easily aspirated away.

Short pulsed laser ophthalmic treatment is typically done in a posterior to anterior progression through the eye because inclusions are created when ocular tissues are photodisrupted. These inclusions are opaque, scatter light, obscure focusing and effectively attenuate the beam, making it difficult to treat “behind” them, such as described in U.S. Pat. No. 5,246,435 by Bille and Schanzlin; as well as U.S. Pat. Appl. Ser. Nos. 2006/0195076 & 2008/0281413 both by one of the present inventors (which are incorporated herein by reference.) Furthermore, laser treating the lens to facilitate its removal creates gas that swells and distorts the lens and potentially moves it due to buoyancy changes. This places the capsule under tension, stretching and distorting it. There can be movement of the lens and capsule in this situation, such as is induced if the aggregate specific gravity of the lens is changed, altering its buoyancy. Thus, the resulting capsulotomy will be caused to be incorrect as the capsule tension relaxes and/or the lens moves during the laser incision, and/or swollen lens material exudes from the capsule as the incision is created. Any of these aforementioned effects lead to an inaccurate and incomplete laser capsulotomy. However, the location, size and shape of the capsulotomy are critical to the patient's visual outcome. Thus, the traditional approach to laser cataract surgery inherently compromises its clinical effectiveness.

Therefore, what is needed are ophthalmic methods, techniques and apparatus for producing a capsulotomy and conditioning the cataractous lens to simplify its removal from the eye that are more accurate and repeatable.

### SUMMARY OF THE INVENTION

The techniques and system disclosed herein provide many advantages. Specifically, the creation of a rapid and precise capsulotomy, intentionally partial or complete, circular or otherwise, followed by the fragmentation of the lens nucleus and cortex using 3-dimensional patterns of ultrashort pulsed laser light. The duration of the procedure and the risk associated with accurately and completely opening the capsule and fragmentation of the hard nucleus are significantly reduced. The removal of a lens dissected into small segments may be performed using patterned laser scanning and using a ultrasonic emulsifier with a conventional phacoemulsification technique or a technique modified to recognize that a segmented lens will likely be more easily removed (i.e., requiring less surgical precision or dexterity) and/or at least with marked reduction in ultrasonic emulsification power, precision and/or duration, as well as providing the ability to do so with smaller and less invasive instruments. There are surgical approaches also enabled the formation of very small and geometrically precise capsulotomies that would be very difficult if not impossible to form using conventional, purely manual techniques. The capsulotomy may further enable greater precision or modifications to conventional ophthalmic procedures as well as enable new procedures. For example, the techniques described herein may be used to facilitate anterior and/or posterior lens removal, implantation of injectable or small foldable IOLs as well as injection of compounds or structures suited to the formation of accommodating IOLs.

In one aspect, a method of making an incision in eye tissue includes generating a beam of light, focusing the beam at a first focal point located at a first depth posterior to the anterior lens capsule and thus within the crystalline lens of the eye of a patient, scanning the beam in a pattern while focused and caused to move anteriorly to incise the lens capsule, thus creating a capsulotomy before performing a complete patterned laser segmentation of the crystalline lens posterior to the laser capsulotomy that is intended to facilitate removal of the crystalline lens.

In another aspect, a method of making an incision in eye tissue includes generating a beam of light, focusing the beam at a first focal point located at a first depth posterior to the anterior lens capsule and thus within the crystalline lens of the eye of a patient, scanning the beam in a pattern while focused and caused to begin the lens conditioning pattern, or a portion of it. Following that partial lens conditioning procedure, the capsulotomy may begin by focusing the beam at a focal point located at a depth posterior to the anterior lens capsule and thus within the crystalline lens of the eye of a patient, scanning the beam in a pattern while focused and caused to move anteriorly to incise the lens capsule, thus creating a capsulotomy prior to completing a complete patterned laser segmentation of the crystalline lens posterior to the laser capsulotomy that is intended to facilitate removal of the crystalline lens. Such an approach may be made to provide an opaque bubble shield within the lens to protect the retina during the remainder of the laser procedure.

Other objects and features of the present invention will become apparent by a review of the specification and appended figures.

### BRIEF DESCRIPTION OF THE DRAWINGS

FIG. 1 is a schematic diagram of the optical beam scanning system.

FIG. 2 is an optical diagram showing an alternative beam combining scheme.

FIG. 3 is a schematic diagram of the optical beam scanning system with an alternative OCT configuration.

FIG. 4 is a schematic diagram of the optical beam scanning system with another alternative OCT combining scheme.

FIG. 5 is an en-face view of an eye lens illustrating the lateral relationship between the capsulotomy and lens conditioning patterns.

FIG. 6 is a flowchart illustrating an embodiment of the present invention.

FIG. 7 is a representation of an elongated capsulotomy pattern, as may be seen when inaccurate imaging/ranging is used.

FIG. 8 is a representation of a targeted capsulotomy pattern, as may be seen when accurate imaging/ranging is used, as described in the present invention.

FIG. 9 is a flowchart illustrating an alternate embodiment of the present invention.

FIG. 10 is a representation of a targeted capsulotomy pattern, as may be seen when accurate imaging/ranging is used, as described in the present invention, along with a lens conditioning pattern.

### DETAILED DESCRIPTION OF THE PREFERRED EMBODIMENTS

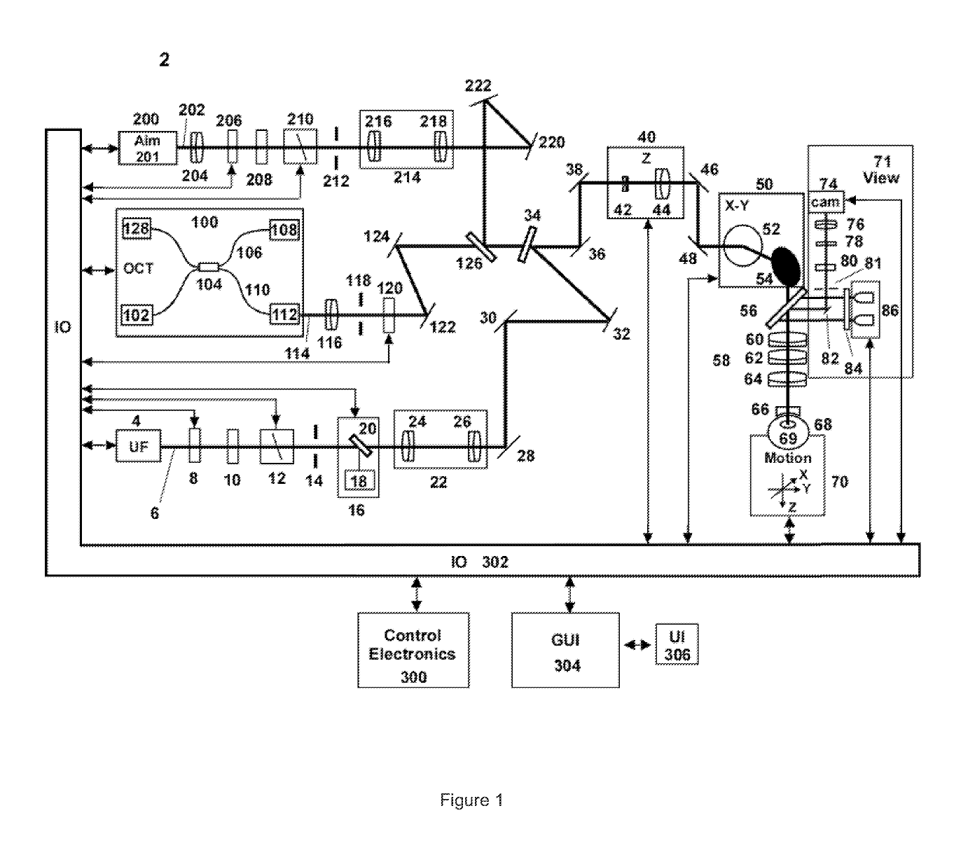

The present invention can be implemented by a system that projects or scans an optical beam into a patient's eye 68, such as system 2 shown in FIG. 1 which includes an ultrafast (UF) light source 4 (e.g. a femtosecond laser). Using this system, a beam may be scanned in a patient's eye in three dimensions: X, Y, Z. Such short pulsed laser light focused into eye tissue will produce dielectric breakdown to cause photodisruption at the focal point, rupturing the tissue in the vicinity of the photo-induced plasma. In this embodiment, the UF wavelength can vary between 1000 nm to 1100 nm and the pulse width can vary from 100 fs to 10000 fs. The pulse repetition frequency can also vary from 10 kHz to 250 kHz. Safety limits with regard to unintended damage to non-targeted tissue bound the upper limit with regard to repetition rate and pulse energy; while threshold energy, time to complete the procedure and stability bound the lower limit for pulse energy and repetition rate. The peak power of the focused spot in the eye 68 and specifically within the crystalline lens 69 and anterior capsule of the eye is sufficient to produce optical breakdown and initiate a plasma-mediated ablation process. Near-infrared wavelengths are preferred because linear optical absorption and scattering in biological tissue is reduced across that spectral range. As an example, laser 4 may be a repetitively pulsed 1035 nm device that produces 500 fs pulses at a repetition rate of 100 kHz and individual pulse energy in the 10 microjoule range.

The laser 4 is controlled by control electronics 300, via an input and output device 302, to create optical beam 6. Control electronics 300 may be a computer, microcontroller, etc. In this example, the entire system is controlled by the controller 300, and data moved through input/output device IO 302. A graphical user interface GUI 304 may be used to set system operating parameters, process user input (UI) 306 on the GUI 304, and display gathered information such as images of ocular structures.

The generated UF light beam 6 proceeds towards the patient eye 68 passing through half-wave plate, 8, and linear polarizer, 10. The polarization state of the beam can be adjusted so that the desired amount of light passes through half-wave plate 8 and linear polarizer 10, which together act as a variable attenuator for the UF beam 6. Additionally, the orientation of linear polarizer 10 determines the incident polarization state incident upon beamcombiner 34, thereby optimizing beamcombiner throughput.

The UF beam proceeds through a shutter 12, aperture 14, and a pickoff device 16. The system controlled shutter 12 ensures on/off control of the laser for procedural and safety reasons. The aperture sets an outer useful diameter for the laser beam and the pickoff monitors the output of the useful beam. The pickoff device 16 includes of a partially reflecting mirror 20 and a detector 18. Pulse energy, average power, or a combination may be measured using detector 18. The information can be used for feedback to the half-wave plate 8 for attenuation and to verify whether the shutter 12 is open or closed. In addition, the shutter 12 may have position sensors to provide a redundant state detection.

The beam passes through a beam conditioning stage 22, in which beam parameters such as beam diameter, divergence, circularity, and astigmatism can be modified. In this illustrative example, the beam conditioning stage 22 includes a 2 element beam expanding telescope comprised of spherical optics 24 and 26 in order to achieve the intended beam size and collimation. Although not illustrated here, an anamorphic or other optical system can be used to achieve the desired beam parameters. The factors used to determine these beam parameters include the output beam parameters of the laser, the overall magnification of the system, and the desired numerical aperture (NA) at the treatment location. In addition, the optical system 22 can be used to image aperture 14 to a desired location (e.g. the center location between the 2-axis scanning device 50 described below). In this way, the amount of light that makes it through the aperture 14 is assured to make it through the scanning system. Pickoff device 16 is then a reliable measure of the usable light.

After exiting conditioning stage 22, beam 6 reflects off of fold mirrors 28, 30, & 32. These mirrors can be adjustable for alignment purposes. The beam 6 is then incident upon beam combiner 34. Beamcombiner 34 reflects the UF beam 6 (and transmits both the OCT 114 and aim 202 beams described below). For efficient beamcombiner operation, the angle of incidence is preferably kept below 45 degrees and the polarization where possible of the beams is fixed. For the UF beam 6, the orientation of linear polarizer 10 provides fixed polarization.

Following the beam combiner 34, the beam 6 continues onto the z-adjust or Z scan device 40. In this illustrative example the z-adjust includes a Galilean telescope with two lens groups 42 and 44 (each lens group includes one or more lenses). Lens group 42 moves along the z-axis about the collimation position of the telescope. In this way, the focus position of the spot in the patient's eye 68 moves along the z-axis as indicated. In general there is a fixed linear relationship between the motion of lens 42 and the motion of the focus. In this case, the z-adjust telescope has an approximate 2× beam expansion ratio and a 1:1 relationship of the movement of lens 42 to the movement of the focus. Alternatively, lens group 44 could be moved along the z-axis to actuate the z-adjust, and scan. The z-adjust is the z-scan device for treatment in the eye 68. It can be controlled automatically and dynamically by the system and selected to be independent or to interplay with the X-Y scan device described next. Mirrors 36 and 38 can be used for aligning the optical axis with the axis of z-adjust device 40.

After passing through the z-adjust device 40, the beam 6 is directed to the x-y scan device by mirrors 46 & 48. Mirrors 46 & 48 can be adjustable for alignment purposes. X-Y scanning is achieved by the scanning device 50 preferably using two mirrors 52 & 54 under the control of control electronics 300, which rotate in orthogonal directions using motors, galvanometers, or any other well known optic moving device. Mirrors 52 & 54 are located near the telecentric position of the objective lens 58 and contact lens 66 combination described below. Tilting these mirrors 52/54 causes them to deflect beam 6, causing lateral displacements in the plane of UF focus located in the patient's eye 68. Objective lens 58 may be a complex multi-element lens element, as shown, and represented by lenses 60, 62, and 64. The complexity of the lens 58 will be dictated by the scan field size, the focused spot size, the available working distance on both the proximal and distal sides of objective 58, as well as the amount of aberration control. An f-theta lens 58 of focal length 60 mm generating a spot size of 10 μm, over a field of 10 mm, with an input beam size of 15 mm diameter is an example. Alternatively, X-Y scanning by scanner 50 may be achieved by using one or more moveable optical elements (e.g. lenses, gratings) which also may be controlled by control electronics 300, via input and output device 302.

The aiming and treatment scan patterns can be automatically generated by the scanner 50 under the control of controller 300. Such patterns may be comprised of a single spot of light, multiple spots of light, a continuous pattern of light, multiple continuous patterns of light, and/or any combination of these. In addition, the aiming pattern (using aim beam 202 described below) need not be identical to the treatment pattern (using light beam 6), but preferably at least defines its boundaries in order to assure that the treatment light is delivered only within the desired target area for patient safety. This may be done, for example, by having the aiming pattern provide an outline of the intended treatment pattern. This way the spatial extent of the treatment pattern may be made known to the user, if not the exact locations of the individual spots themselves, and the scanning thus optimized for speed, efficiency and accuracy. The aiming pattern may also be made to be perceived as blinking in order to further enhance its visibility to the user.

An optional contact lens 66, which can be any suitable ophthalmic lens, can be used to help further focus the optical beam 6 into the patient's eye 68 while helping to stabilize eye position. The positioning and character of optical beam 6 and/or the scan pattern the beam 6 forms on the eye 68 may be further controlled by use of an input device such as a joystick, or any other appropriate user input device (e.g. GUI 304) to position the patient and/or the optical system.

The UF laser 4 and controller 300 can be set to target the surfaces of the targeted structures in the eye 68 and ensure that the beam 6 will be focused where appropriate and not unintentionally damage non-targeted tissue. Imaging modalities and techniques described herein, such as for example, Optical Coherence Tomography (OCT), Purkinje imaging, Scheimpflug imaging, or ultrasound may be used to determine the location and measure the thickness of the lens and lens capsule to provide greater precision to the laser focusing methods, including 2D and 3D patterning. Laser focusing may also be accomplished using one or more methods including direct observation of an aiming beam, Optical Coherence Tomography (OCT), Purkinje imaging, Scheimpflug imaging, ultrasound, or other known ophthalmic or medical imaging modalities and/or combinations thereof. In the embodiment of FIG. 1, an OCT device 100 is described, although other modalities are within the scope of the present invention. An OCT scan of the eye will provide information about the axial location of the anterior and posterior lens capsule, the boundaries of the cataract nucleus, as well as the depth of the anterior chamber. This information is then be loaded into the control electronics 300, and used to program and control the subsequent laser-assisted surgical procedure. The information may also be used to determine a wide variety of parameters related to the procedure such as, for example, the upper and lower axial limits of the focal planes used for cutting the lens capsule and segmentation of the lens cortex and nucleus, and the thickness of the lens capsule among others.

The OCT device 100 in FIG. 1 includes a broadband or a swept light source 102 that is split by a fiber coupler 104 into a reference arm 106 and a sample arm 110. The reference arm 106 includes a module 108 containing a reference reflection along with suitable dispersion and path length compensation. The sample arm 110 of the OCT device 100 has an output connector 112 that serves as an interface to the rest of the UF laser system. The return signals from both the reference and sample arms 106, 110 are then directed by coupler 104 to a detection device 128, which employs either time domain, frequency or single point detection techniques. In FIG. 1, a frequency domain technique is used with an OCT wavelength of 920 nm and bandwidth of 100 nm.

Exiting connector 112, the OCT beam 114 is collimated using lens 116. The size of the collimated beam 114 is determined by the focal length of lens 116. The size of the beam 114 is dictated by the desired NA at the focus in the eye and the magnification of the beam train leading to the eye 68. Generally, OCT beam 114 does not require as high an NA as the UF beam 6 in the focal plane and therefore the OCT beam 114 is smaller in diameter than the UF beam 6 at the beamcombiner 34 location. Following collimating lens 116 is aperture 118 which further modifies the resultant NA of the OCT beam 114 at the eye. The diameter of aperture 118 is chosen to optimize OCT light incident on the target tissue and the strength of the return signal. Polarization control element 120, which may be active or dynamic, is used to compensate for polarization state changes which may be induced by individual differences in corneal birefringence, for example. Mirrors 122 & 124 are then used to direct the OCT beam 114 towards beamcombiners 126 & 34. Mirrors 122 & 124 may be adjustable for alignment purposes and in particular for overlaying of OCT beam 114 to UF beam 6 subsequent to beamcombiner 34. Similarly, beamcombiner 126 is used to combine the OCT beam 114 with the aim beam 202 described below.

Once combined with the UF beam 6 subsequent to beamcombiner 34, OCT beam 114 follows the same path as UF beam 6 through the rest of the system. In this way, OCT beam 114 is indicative of the location of UF beam 6. OCT beam 114 passes through the z-scan 40 and x-y scan 50 devices then the objective lens 58, contact lens 66 and on into the eye 68. Reflections and scatter off of structures within the eye provide return beams that retrace back through the optical system, into connector 112, through coupler 104, and to OCT detector 128. These return back reflections provide the OCT signals that are in turn interpreted by the system as to the location in X, Y Z of UF beam 6 focal location.

OCT device 100 works on the principle of measuring differences in optical path length between its reference and sample arms. Therefore, passing the OCT through z-adjust 40 does not extend the z-range of OCT system 100 because the optical path length does not change as a function of movement of 42. OCT system 100 has an inherent z-range that is related to the detection scheme, and in the case of frequency domain detection it is specifically related to the spectrometer and the location of the reference arm 106. In the case of OCT system 100 used in FIG. 1, the z-range is approximately 1-2 mm in an aqueous environment. Extending this range to at least 4 mm involves the adjustment of the path length of the reference arm within OCT system 100. Passing the OCT beam 114 in the sample arm through the z-scan of z-adjust 40 allows for optimization of the OCT signal strength. This is accomplished by focusing the OCT beam 114 onto the targeted structure while accommodating the extended optical path length by commensurately increasing the path within the reference arm 106 of OCT system 100.

Because of the fundamental differences in the OCT measurement with respect to the UF focus device due to influences such as immersion index, refraction, and aberration, both chromatic and monochromatic, care must be taken in analyzing the OCT signal with respect to the UF beam focal location. A calibration or registration procedure as a function of X, Y Z should be conducted in order to match the OCT signal information to the UF focus location and also to the relate to absolute dimensional quantities.

Observation of an aim beam may also be used to assist the user to directing the UF laser focus. Additionally, an aim beam visible to the unaided eye in lieu of the infrared OCT and UF beams can be helpful with alignment provided the aim beam accurately represents the infrared beam parameters. An aim subsystem 200 is employed in the configuration shown in FIG. 1. The aim beam 202 is generated by a an aim beam light source 201, such as a helium-neon laser operating at a wavelength of 633 nm. Alternatively a laser diode in the 630-650 nm range could be used. The advantage of using the helium neon 633 nm beam is its long coherence length, which would enable the use of the aim path as a laser unequal path interferometer (LUPI) to measure the optical quality of the beam train, for example.

Once the aim beam light source generates aim beam 202, the aim beam 202 is collimated using lens 204. The size of the collimated beam is determined by the focal length of lens 204. The size of the aim beam 202 is dictated by the desired NA at the focus in the eye and the magnification of the beam train leading to the eye 68. Generally, aim beam 202 should have close to the same NA as UF beam 6 in the focal plane and therefore aim beam 202 is of similar diameter to the UF beam at the beamcombiner 34 location. Because the aim beam is meant to stand-in for the UF beam 6 during system alignment to the target tissue of the eye, much of the aim path mimics the UF path as described previously. The aim beam 202 proceeds through a half-wave plate 206 and linear polarizer 208. The polarization state of the aim beam 202 can be adjusted so that the desired amount of light passes through polarizer 208. Elements 206 & 208 therefore act as a variable attenuator for the aim beam 202. Additionally, the orientation of polarizer 208 determines the incident polarization state incident upon beamcombiners 126 and 34, thereby fixing the polarization state and allowing for optimization of the beamcombiners' throughput. Of course, if a semiconductor laser is used as aim beam light source 200, the drive current can be varied to adjust the optical power.

The aim beam 202 proceeds through a shutter 210 and aperture 212. The system controlled shutter 210 provides on/off control of the aim beam 202. The aperture 212 sets an outer useful diameter for the aim beam 202 and can be adjusted appropriately. A calibration procedure measuring the output of the aim beam 202 at the eye can be used to set the attenuation of aim beam 202 via control of polarizer 206.

The aim beam 202 next passes through a beam conditioning device 214. Beam parameters such as beam diameter, divergence, circularity, and astigmatism can be modified using one or more well known beaming conditioning optical elements. In the case of an aim beam 202 emerging from an optical fiber, the beam conditioning device 214 can simply include a beam expanding telescope with two optical elements 216 and 218 in order to achieve the intended beam size and collimation. The final factors used to determine the aim beam parameters such as degree of collimation are dictated by what is necessary to match the UF beam 6 and aim beam 202 at the location of the eye 68. Chromatic differences can be taken into account by appropriate adjustments of beam conditioning device 214. In addition, the optical system 214 is used to image aperture 212 to a desired location such as a conjugate location of aperture 14.

The aim beam 202 next reflects off of fold mirrors 222 & 220, which are preferably adjustable for alignment registration to UF beam 6 subsequent to beam combiner 34. The aim beam 202 is then incident upon beam combiner 126 where the aim beam 202 is combined with OCT beam 114. Beamcombiner 126 reflects the aim beam 202 and transmits the OCT beam 114, which allows for efficient operation of the beamcombining functions at both wavelength ranges. Alternatively, the transmit and reflect functions of beamcombiner 126 can be reversed and the configuration inverted. Subsequent to beamcombiner 126, aim beam 202 along with OCT beam 114 is combined with UF beam 6 by beamcombiner 34.

A device for imaging the target tissue on or within the eye 68 is shown schematically in FIG. 1 as imaging system 71. Imaging system includes a camera 74 and an illumination light source 86 for creating an image of the target tissue. The imaging system 71 gathers images which may be used by the system controller 300 for providing pattern centering about or within a predefined structure. The illumination light source 86 for the viewing is generally broadband and incoherent. For example, light source 86 can include multiple LEDs as shown. The wavelength of the viewing light source 86 is preferably in the range of 700 nm to 750 nm, but can be anything which is accommodated by the beamcombiner 56, which combines the viewing light with the beam path for UF beam 6 and aim beam 202 (beamcombiner 56 reflects the viewing wavelengths while transmitting the OCT and UF wavelengths). The beamcombiner 56 may partially transmit the aim wavelength so that the aim beam 202 can be visible to the viewing camera 74. Optional polarization element 84 in front of light source 86 can be a linear polarizer, a quarter wave plate, a half-wave plate or any combination, and is used to optimize signal. A false color image as generated by the near infrared wavelength is acceptable.

The illumination light from light source 86 is directed down towards the eye using the same objective lens 58 and contact lens 66 as the UF and aim beam 6, 202. The light reflected and scattered off of various structures in the eye 68 are collected by the same lenses 58 & 66 and directed back towards beamcombiner 56. There, the return light is directed back into the viewing path via beam combiner and mirror 82, and on to camera 74. Camera 74 can be, for example but not limited to, any silicon based detector array of the appropriately sized format. Video lens 76 forms an image onto the camera's detector array while optical elements 80 & 78 provide polarization control and wavelength filtering respectively. Aperture or iris 81 provides control of imaging NA and therefore depth of focus and depth of field. A small aperture provides the advantage of large depth of field which aids in the patient docking procedure. Alternatively, the illumination and camera paths can be switched. Furthermore, aim light source 200 can be made to emit in the infrared which would not directly visible, but could be captured and displayed using imaging system 71.

Coarse adjust registration is usually needed so that when the contact lens 66 comes into contact with the cornea, the targeted structures are in the capture range of the X, Y scan of the system. Therefore a docking procedure is preferred, which preferably takes in account patient motion as the system approaches the contact condition (i.e. contact between the patient's eye 68 and the contact lens 66. The viewing system 71 is configured so that the depth of focus is large enough such that the patient's eye 68 and other salient features may be seen before the contact lens 66 makes contact with eye 68.

Preferably, a motion control system 70 is integrated into the overall control system 2, and may move the patient, the system 2 or elements thereof, or both, to achieve accurate and reliable contact between contact lens 66 and eye 68. Furthermore, a vacuum suction subsystem and flange may be incorporated into system 2, and used to stabilize eye 68. The alignment of eye 68 to system 2 via contact lens 66 may be accomplished while monitoring the output of imaging system 71, and performed manually or automatically by analyzing the images produced by imaging system 71 electronically by means of control electronics 300 via IO 302. Force and/or pressure sensor feedback may also be used to discern contact, as well as to initiate the vacuum subsystem.

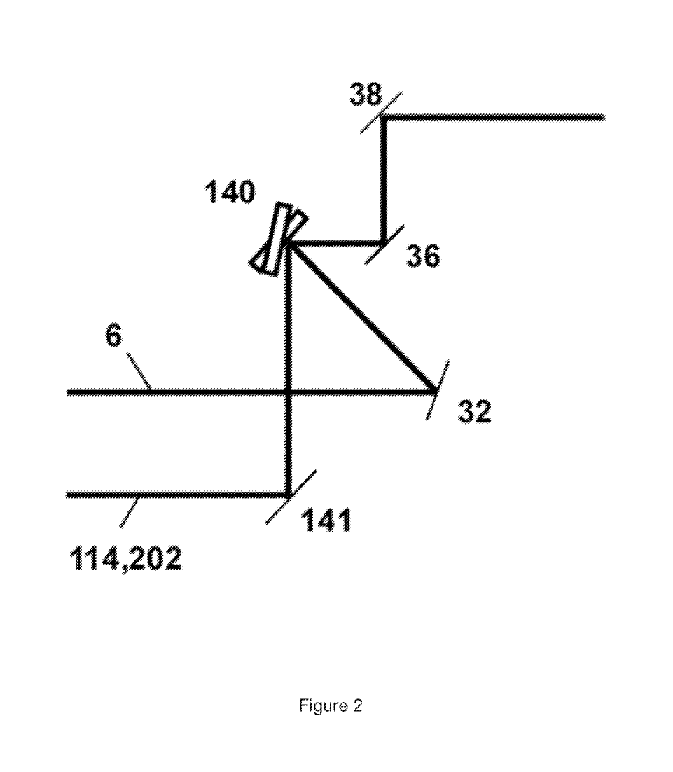

An alternative beamcombining configuration is shown in the alternate embodiment of FIG. 2. For example, the passive beamcombiner 34 in FIG. 1 can be replaced with an active combiner 140 in FIG. 2. The active beamcombiner 34 can be a moving or dynamically controlled element such as a galvanometric scanning mirror, as shown. Active combiner 140 changes it angular orientation in order to direct either the UF beam 6 or the combined aim and OCT beams 202,114 towards the scanner 50 and eventually eye 68 one at a time. The advantage of the active combining technique is that it avoids the difficulty of combining beams with similar wavelength ranges or polarization states using a passive beam combiner. This ability is traded off against the ability to have simultaneous beams in time and potentially less accuracy and precision due to positional tolerances of active beam combiner 140.

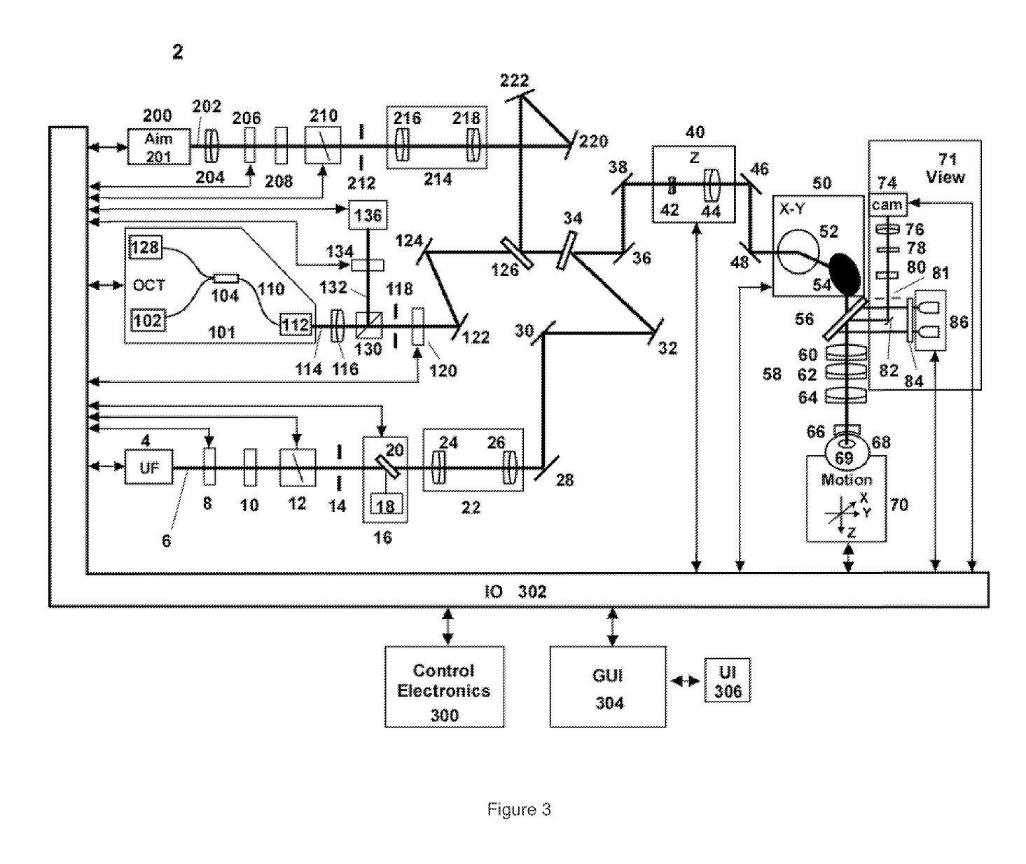

Another alternate embodiment is shown in FIG. 3 which is similar to that of FIG. 1 but utilizes an alternate approach to OCT 100. In FIG. 3, OCT 101 is the same as OCT 100 in FIG. 1, except that the reference arm 106 has been replaced by reference arm 132. This free-space OCT reference arm 132 is realized by including beamsplitter 130 after lens 116. The reference beam 132 then proceeds through polarization controlling element 134 and then onto the reference return module 136. The reference return module 136 contains the appropriate dispersion and path length adjusting and compensating elements and generates an appropriate reference signal for interference with the sample signal. The sample arm of OCT 101 now originates subsequent to beamsplitter 130. The potential advantages of this free space configuration include separate polarization control and maintenance of the reference and sample arms. The fiber based beam splitter 104 of OCT 101 can also be replaced by a fiber based circulator. Alternately, both OCT detector 128 and beamsplitter 130 might be moved together as opposed to reference arm 136.

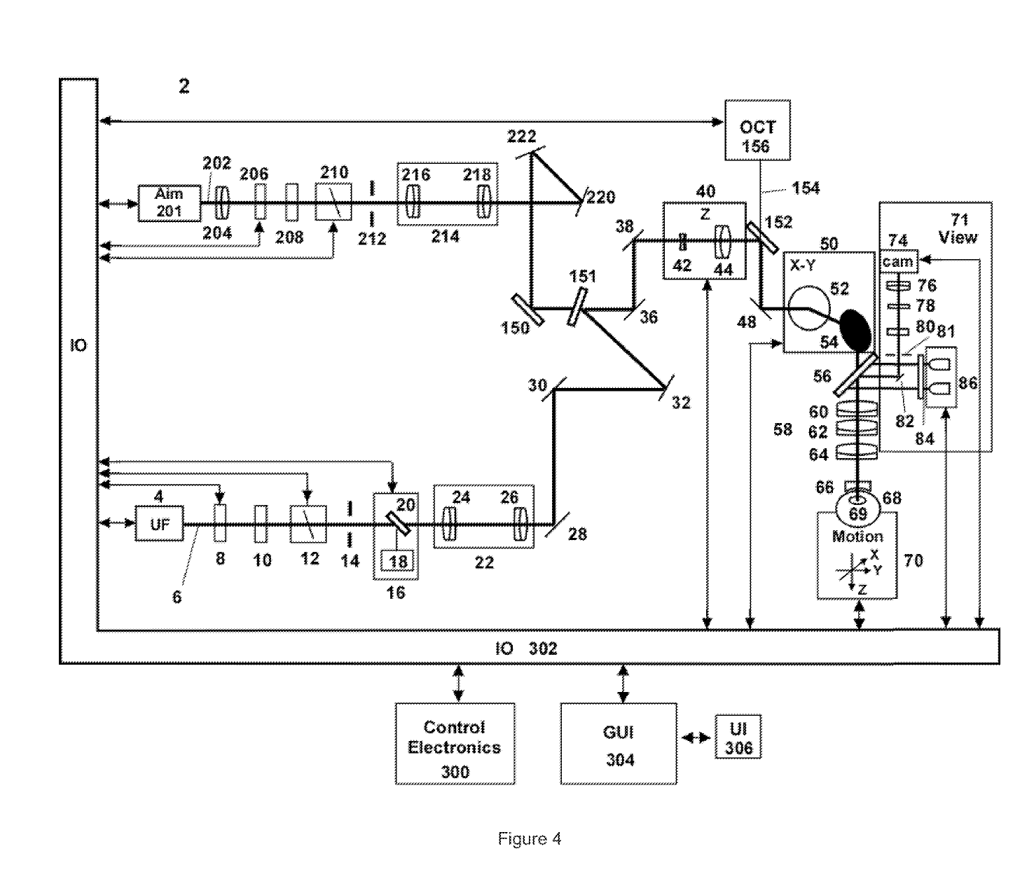

FIG. 4 shows another alternative embodiment for combining OCT beam 114 and UF beam 6. In FIG. 4, OCT 156 (which can include either of the configurations of OCT 100 or 101) is configured such that its OCT beam 154 is coupled to UF beam 6 after the z-scan 40 using beamcombiner 152. In this way, OCT beam 154 avoids using the z-adjust. This allows the OCT 156 to possibly be folded into the beam more easily and shortening the path length for more stable operation. This OCT configuration is at the expense of an optimized signal return strength as discussed with respect to FIG. 1. There are many possibilities for the configuration of the OCT interferometer, including time and frequency domain approaches, single and dual beam methods, swept source, etc, as described in U.S. Pat. Nos. 5,748,898; 5,748,352; 5,459,570; 6,111,645; and 6,053,613 (which are incorporated herein by reference.)

The laser 10 and controller 12 can be set to locate the surface of the capsule and ensure that the beam will be focused on the lens capsule at all points of the desired opening. Imaging modalities and techniques described herein, such as for example, Optical Coherence Tomography (OCT), Scheimpflug, Confocal Microscopy, or ultrasound, may be used to determine the shape, geometry, perimeter, boundaries, and/or 3-dimensional location of the lens and lens capsule to provide greater precision to the laser focusing methods, including 2D and 3D patterning. Laser focusing may also be accomplished using one or more methods including direct observation of an aiming beam, or other known ophthalmic or medical imaging modalities and combinations thereof, such as but not limited to those defined above.

OCT imaging of the anterior chamber can be performed on the lens using the same laser and/or the same scanner used to produce the patterns for cutting. This scan will provide information about the axial location and shape (and even thickness) of the anterior and posterior lens capsule, the boundaries of the cataract nucleus, as well as the depth of the anterior chamber. This information may then be loaded into the laser 3-D scanning system or used to generate a 3 dimensional representation of the anterior chamber and lens of the eye, and used to define the patterns used in the surgical procedure.

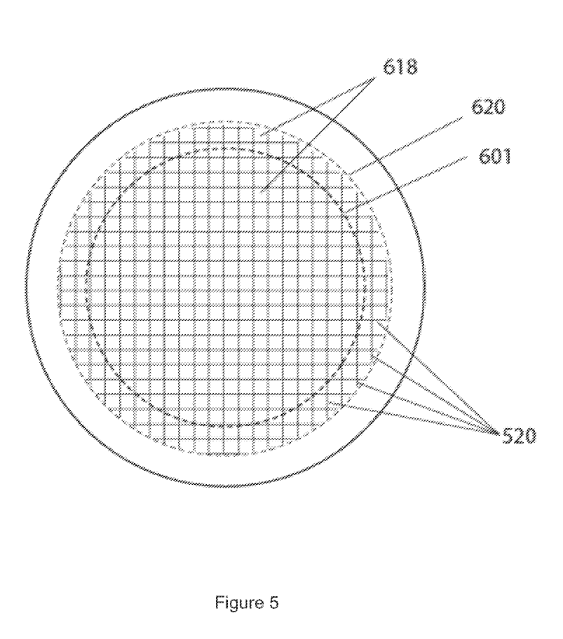

FIG. 5 shows an en-face view of exemplary patterns for capsulotomy and lens conditioning, with an array of rectangular planar cuts 520 (i.e. crossing array of rows and columns of cuts) creating pattern 620 to facilitate removal of lens 69 by segmenting it into rectangular sub-elements 618. The capsulotomy 601 is shown as (dashed line) being smaller in lateral extent than the lens conditioning pattern 620, but need not be. The width of a single section 618 should be made to be smaller than the inner-diameter of the phacoemulsification tip 541, typically but not limited to inner-diameters between 1.1 mm and 0.25 mm. Although liberated gases tend to migrate along the lamella and “pneumodissect” the lens, planes orthogonal to those shown may also be cut into lens 69 to create still smaller segments and further assist with lens removal, especially with very hard nuclei.

For anterior and posterior capsulotomy, the scanning patterns can be stepped circular and spiral, with a vertical step or pitch similar to the length of the rupture zone. For segmentation of the eye lens 69, the patterns can be linear, planar, radial, radial segments, circular, spiral, curvilinear and combinations thereof including patterning in two and/or three dimensions. Scans can be continuous straight or curved lines, or one or more overlapping or spaced apart spots and/or line segments. In addition, these and other 2D and 3D patterns may be used in combination with OCT to obtain additional imaging, anatomical structure or make-up (i.e., tissue density) or other dimensional information about the eye including but not limited to the lens, the cornea, the retina and as well as other portions of the eye that serve to define boundaries of the anatomical target and subsequently of the laser shot pattern(s).

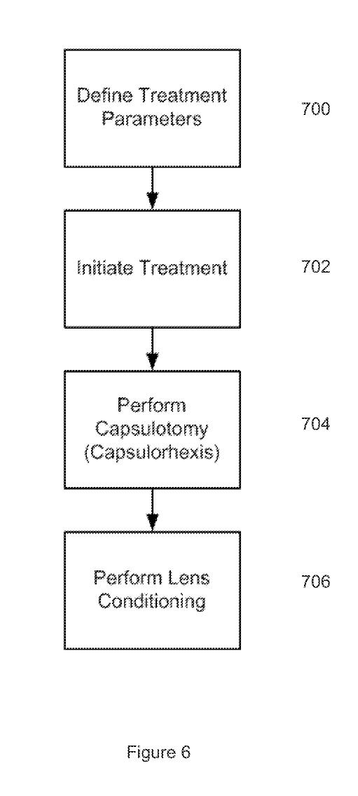

FIG. 6 is a flowchart detailing the basic steps of one embodiment of the present inventive method. In this embodiment, treatment parameters 700 are defined, followed by the initiate treatment 702 step wherein the perform capsulotomy 704 step is completed prior to perform lens conditioning 706 step.

When performing both a capsulotomy and a lens conditioning procedure (e.g. lens softening or segmentation) using laser energy, the standard wisdom is that one needs to cut from posterior to anterior in order to avoid transmission through the opacity that results from the inclusions created by plasma-mediated laser ablation. For these reasons, it has been believed that it is best to condition the lens first, and then perform the capsulotomy on the anterior surface of the capsule to access the conditioned lens. However, we have discovered that it is best to perform the capsulotomy before conditioning the lens to any great extent, even though it is counterintuitive to do so. This is due to the fact that a significant amount of gas is created within the lens during the conditioning process which causes it to swell within the unopened capsule. This places the capsule under tension, stretching it, distorting it, and moving it by changing its aggregate specific gravity and buoyancy within the eye. The resulting capsulorhexis will be incorrect as the capsule tension relaxes and/or swollen lens material exudes from the capsule as the incision is created. This is unacceptable, as the size and shape of the capsulorhexis is critical to the patient's visual outcome.

In order to minimize the negative effects of these resultant inclusions on the subsequent lens conditioning, it is best to identify and target the capsule directly rather than simply providing an extended capsulotomy pattern that assures one will incise the capsule as it is traversed without specific knowledge of its location. Therefore, instead of using beam delivery parameters that produce an extended capsulotomy pattern in the x, y and z axes, it is preferred to implement an integrated ranging/imaging scheme that locates the capsule in order to accurately place the capsulotomy pattern, such as those described herein.

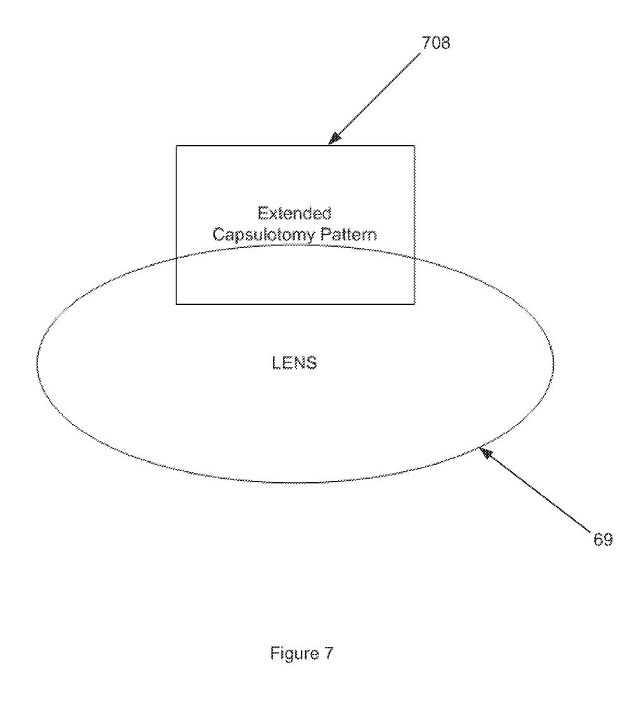

FIG. 7 illustrates an embodiment wherein the capsulotomy pattern is extended 708 to compensate for inadequate ranging/imaging of the lens 69. While still suitable for use with the present invention, this situation may compromise the effectiveness of the lens segmentation patterns due to the presence of bubbles in the anterior chamber that may serve to perturb the treatment beam.

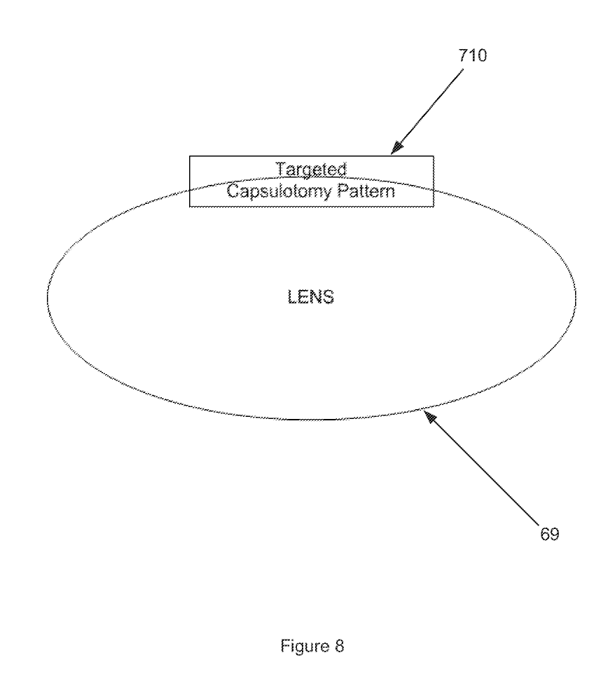

FIG. 8 shows a more optimized targeted capsulotomy pattern 710 that minimizes the amount of bubbles created. The axial extent of capsulotomy pattern 710 is limited to the vicinity of the anterior capsule/surface of lens 69 by virtue of accurate ranging/imaging.

The integrated ranging/imaging configuration may include a combination of one or more of the following non-limiting examples: OCT, confocal imaging, ultrasound detection, Purkinje imaging, and/or Scheimpflug imaging for 3-dimensional lens location, as described above; or video for 2-dimensional and/or stereoscopic lens location.

This approach provides the ability to accurately locate and then use the laser to incise the anterior lens capsule, thus minimizing the number of laser pulses required to produce a complete capsulotomy. This allows for the capsulotomy to be cut prior to conditioning the lens it contains and thus provides means for a more accurately sized, shaped and complete incision. Minimizing the axial extent of the capsulotomy pattern due to accurate imaging of the lens and/or lens capsule also minimizes the amount of resultant opacity that would otherwise hinder the performance of the laser beam that later traverses it in order to treat the lens material beyond, including any bubbles formed by gas liberated from the procedure that may settle in the anterior chamber of the eye. Such bubbles may come from laser pulses disposed within the lens during the beginning of the capsulotomy pattern that migrate out once the capsule integrity is breached by the incision, or ultimately from the laser pulses disposed within the aqueous humor itself once the beam has traversed the capsule and the pattern is completed. These bubbles perturb the optical performance of the treatment beam and thereby hinder its effect in tissue. However, the requisite depth, and this the number of pulses required, of the capsulotomy pattern is minimized when accurate imaging and/or ranging is utilized to locate the anterior lens capsule and/or anterior lens surface.

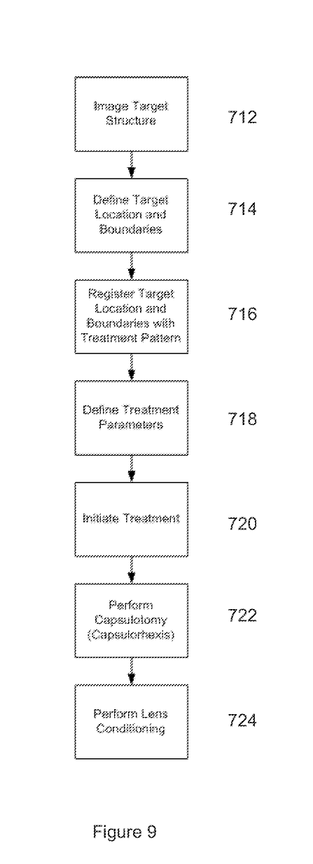

FIG. 9 illustrates a more detailed flow diagram of one embodiment of the present inventive method. In this embodiment, an Image Target Structure(s) 712 step is performed initially, followed by definition of target location and boundaries 714, registration of target location and boundaries with the treatment pattern 716, definition of treatment parameters 718, initiation of treatment 720, performance of capsulotomy (capsulorhexis) 722, and performance of lens conditioning intervention 724.

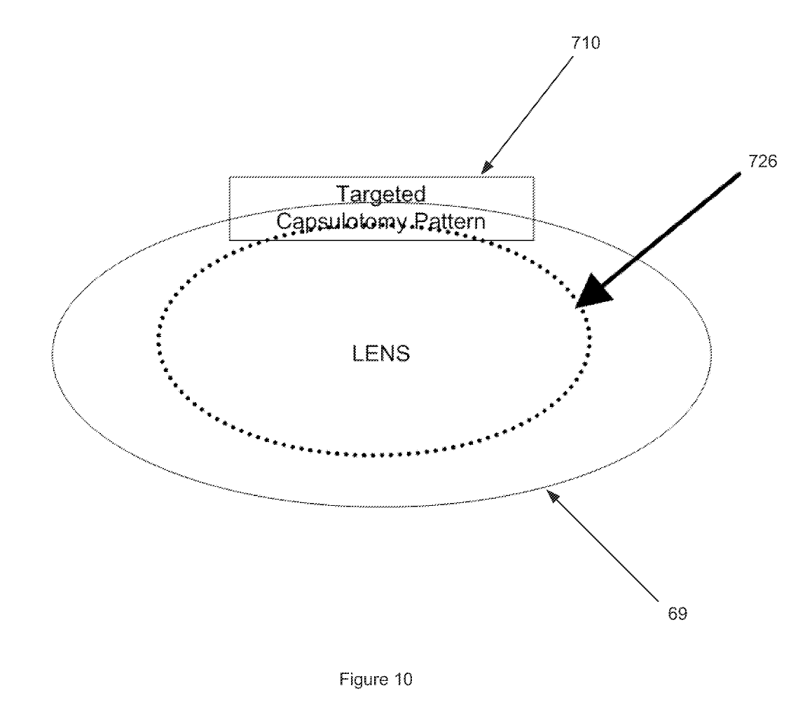

The embodiment illustrated in FIG. 10 is similar to that of FIG. 8 with the addition of lens conditioning pattern 726. As described previously, lens conditioning pattern 726 may be created entirely subsequent to capsulotomy pattern 710, or piecewise with capsulotomy pattern 710, being completed in between parts of lens pattern 726, as was described above.

It is to be understood that the present invention is not limited to the embodiments described above and illustrated herein, but encompasses any and all variations explicitly and implicitly derived therefrom. For example, the lens conditioning may be made in multiple steps, with the capsulotomy occurring between them to accomplish the intended goal. Although not shown in the figures, multiple imaging steps may also be employed in between treatment steps to account for any changes in position and/or size due to treatment and further insure the accurate disposition of laser energy in the target tissue.

1. A method of treating the crystalline lens of the eye of a patient, comprising:
a. acquiring image information pertinent to the lens of the patient;
b. forming a capsolutomy treatment pattern with laser-based incisions; and
c. forming a lens-conditioning treatment pattern with laser-based incisions after the capsulotomy has been formed.

2. A method of treating the crystalline lens of the eye of a patient, comprising:
a. acquiring image information pertinent to the lens of the patient;
b. form at least a portion of a lens-conditioning treatment pattern using laser-based incisions;
c. forming a capsolutomy treatment pattern with laser-based incisions; and
d. completing formation of the lens-conditioning treatment pattern.

3. A method of treating the crystalline lens of the eye of a patient, comprising:
a. acquiring image information pertinent to the lens of the patient;
b. form at least a portion of a lens-conditioning treatment pattern using laser-based incisions;
c. acquiring additional image information pertinent to the lens of the patient;
d. forming a capsolutomy treatment pattern with laser-based incisions;
e. acquiring further image information pertinent to the lens of the patient; and
f. completing formation of the lens-conditioning treatment pattern.

4. A method of treating the crystalline lens of the eye of a patient, comprising:
a. acquiring image information pertinent to the lens of the patient;
b. forming a capsolutomy treatment pattern with laser-based incisions;
c. acquiring additional image information pertinent to the lens of the patient;
d. form a lens-conditioning treatment pattern using laser-based incisions.
